(19) United States
(12) Patent Application Publication
(10) Pub. No.: US 2014/0196171 A1
(43) Pub. Date: Jul. 10, 2014

(54) PLANT PROMOTERS INDUCED BY HYDROLOGICAL SHORTAGE AND USE THEREOF

(71) Applicant: Universidad de Talca, Talca (CL)

(72) Inventors: Simon Ruiz Lara, Talca (CL); José Antonio Casaretto Vargas, Talca (CL); Analia Antonella Espinoza Contreras, Rengo (CL)

(73) Assignee: Universidad de Talca, Talca (CL)

(21) Appl. No.: 14/089,960

(22) Filed: Nov. 26, 2013

(30) Foreign Application Priority Data
Nov. 27, 2012 (CL) 3316-2012

(51) Int. Cl. C12N 15/82

(52) U.S. Cl. CPC C12N 15/8241
USPC 800/278; 536/24.1; 435/320.1; 435/419; 435/91.2

(57) ABSTRACT

A nucleotide promoter sequence permits regulation of gene expression in plants including at least 80% of identity with sequence or a portion of promoter sequence of genes Atlg05340 or Atlg80160 of Arabidopsis. A method obtains a plant genetically modified with such a promoter nucleotide sequence and a method obtains promoter regions of genes Atlg05340 or Atlg80160 of Arabidopsis.

### TECHNICAL FIELD

This disclosure relates to the field of agricultural biotechnology and molecular biology in plants, especially to the regulation of gene expression in plants. Specifically, this disclosure relates to isolation, characterization and use of promoter regions that permit the expression of genes in plants to improve the growth and development of plants when they are in conditions of drought or ground salinity. Additionally, the disclosure relates to plants genetically modified showing a gene of interest fused with said promoter sequences.

### BACKGROUND

Practically, all agricultural areas are subjected to lack of water due to climate variations or socioeconomic problems that limit water resources. Therefore, it is of great importance and interest to obtain crops with better tolerance to hydrological stress. In the agricultural field, this is generally achieved by conventional improvement, a process that is very laborious and not always efficient, depending on the vegetal species. The possibility of transference of any genetic element to the vegetal makes possible generation of crops with diverse qualities among them with a greater tolerance to stress. Strategies that achieve improvements in tolerance to drought will permit greater production in areas affected by said stress or obtain adequate performance with less use of water.

The regulation of gene expression is fundamental for growth, development and survival of vegetal species. Adverse environmental factors affect gene expression depending on the nutritional state and the stage of development in plants. Plants respond to hydrological stress, with biochemical and physiological modifications that may be implicated in tolerance and adaptation mechanisms. Diverse genes have been described for their capacity to respond to environmental stimulus such as droughts, salinity or extreme temperatures. Variability and gene expression control is due mainly to the presence of regulating elements present in the regulating or “promoter” region in the gene (Kilian et al., 2007, Plant J. 50(2): 347-363).

DNA sequence expression (transcription) in a vegetal organism depends then on the promoter sequence connected operatively to DNA sequences and they are functional inside the vegetal. Election of the promoter sequence will determine when, where and how intense the gene DNA sequence of interest will be transcribed inside the organism. When the expression (transcription) is desired to be continuous in all tissues of a plant, constitutive promoters are used. When the expression is desired to occur in specific tissues and organs, specific promoters can be used for each tissue. When gene expression is desired to occur as a response to a stimulus, inducible promoters are used and contain regulating elements that cause expression only in the presence of a stimulus. Genetically altering plants through the use of techniques of genetic engineering and produce thereby a plant with useful features requires availability of efficient promoters. For example, U.S. Pat. No. 7,314,757, U.S. 2009/0229014, U.S. 2011/0010796 and US 2009/0282582 can be cited, which describe plant promoters inducible by hydrological stress.

Promoters contain specific sequences capable of directing transcription, recognition sites (elements in cis) by transcription activator or inhibitor proteins (trans elements). Transcription factors are proteins, of nuclear localization without enzymatic function, they interact with cis elements by recognizing small sequences conserved in DNA, and they modulate the expression of one or more genes. Transcription factors have been classified in families and sub-families according to homology in their DNA binding domains (Mace et al., 2006, Bioinformatics 22(14): e323-e331).

Several studies have described binding elements and transcription factors involved in response to adverse environmental conditions. Hydrological stress and salinity can activate two types of response: a response modulated by the presence of Abscisic acid hormone (ABA) and an independent path of this phytohormone (Shinozaki y Yamaguchi-Shinozaki, 2007, J. Exp. Bot. 58: 221-7; Yamaguchi-Shinozaki y Shinozaki, 2005, Trend Plant Sci. 10(2): 88-94). The system depending on ABA involves transcription factors with binding sites to elements of response to ABA (or ABREs) called AREBs/ABFs, other called MYC, MYB and HDZIP (Fujita et al. 2011, J. Plant Res. 124(4): 509-25; Du et al. 2009 Biochemistry (Mosc) 74(1): 1-11; Dai et al., 2007, Plant Physiol. 143(4): 1739-1751) and others that frequently function in independent paths to ABA and respond to dehydration designed DREB/CBF, NAC, ZFHD (Agarwal et al. 2006, Plant Cell Rep. 25(12): 1263-74; Lata et al. 2011, J. Exp. Bot. 62(14): 4731-48; Mizoi et al. 2012, Biochim. Biophys. Acta 1819(2): 86-96; Nakashima et al. 2012, Biochim. Biophys. Acta 1819(2): 97-103). The response to stress caused by cold is modulated independently to ABA through certain factors CBF/DREB (Lata et al. 2011, J. Exp. Bot. 62(14): 4731-48). Therefore, components present in the promoter and associated factors can help to improve strategies used for the heterologous gene expression (from other species).

In strategies to improve plants tolerance to hydrological stress, genetic engineering has permitted generation of plants with new characteristics through the incorporation of genes associated with stress. However, frequently, plants are obtained with phenotypic characteristics not desired, especially when transgene expression is controlled by a promoter of constitutive activation as in the case of cauliflower virus mosaic 35S (CaMV35S). Commonly, phenotypes include growth inhibition, chlorosis and necrosis (Gilmour et al., 2000, Plant Physiol. 124(4): 1854-1865; Chen et al., 2006, J. Exp. Bot. 57(9): 2101-2110; Pino et al., 2007, Plant Biotechnol. J. 5(5): 591-604; Park et al., 2009, Plant Cell Physiol. 50(4): 869-878). An alternative to this problem is the use of specific promoters that control gene expression at some stage of development, specifically in a tissue or, in the case herein, in conditions when a response to stress is required.

Diverse studies have described the use of specific promoters associated with organs or tissues such a as pollen (Bernd-Souza et al., 2000, Genet. Mol. Biol. 23: 435-443), seeds (Zakharov et al., 2004, J. Exp. Bot. 55(402): 1463-1471), phloem (Shi et al., 1994, J. Exp. Bot. 45(5): 623-631); wounds-inducible promoters (Kock et al., 2004, Planta 219(2): 233-242); associated to abiotic stress (Su et al., 2006, J. Exp. Bot. 57(5): 1129-1135), among others. One of the most known cases is the promoter of gene RD29A of Arabidopsis that is induced significantly by cold, dehydration, salinity and ABA (Yamaguchi-Shinozaki y Shinozaki, 1993, Mol. Gen. Gen. 236(2): 331-340). Promoter RD29A is characterized by presenting at least two types of regulating elements. One of them, called DRE, is activated in the rapid response to stress by low temperatures, dehydration and salt through ways of signaling independent to ABA, and others ABREs that respond to ABA signaling. The latter, is involved in the second induction of RD29A, after accumulation of ABA in conditions of hydrological stress caused by drought or salinity (Narusaka et al., 2003, Plant J. 34(2): 137-148). An example is the case of a variety of potato that was genetically modified with three genes CBF of Arabidopsis (AtCBF1-3) under control of promoter CaMV35S and RD29A. These plants CAMV35S-AtCBF increased their tolerance to freezing at 2° C. However, they exhibited a diminution in foliar area, delay in the flowering period and less production of tubercles. On the other hand, plants transformed with the genetic construction RD29A-AtCBF exhibited equal tolerance to low temperatures than plants of constitutive expression and equal production of tubercles than wild plants (Pino et al., 2007, Plant Biotechnol. J. 5(5): 591-604), which suggests that the use of inducible promoters can provide benefits for controlled gene expression without affecting negatively the important agronomical characteristics.

### SUMMARY

We provide promoter sequences that permit expression of a gene of interest in plants when they are found in conditions of hydrological shortage.

Advantages are that regulator nucleotide sequences of gene transcription provide a low basal expression (in optimal conditions of water resource for plants) differently from promoters of constitutive activity such as CaMV35S or Ubi1 that constantly cause the vegetal tissue to synthesize the codified proteins by the gene regulated by these sequences. This demand of unnecessary consumption of energy (source of carbon and nitrogen) affects in the end the normal growth and species development.

Our regulator nucleotide sequences exhibit an activation of its promoter activity in conditions of hydrological shortage, as shown below. These levels of activation can become equal or higher than strong constitutive promoters such as CaMV35S or other inducible ones of public knowledge as RD29B.

Our promoter nucleotide sequences behave in an efficient way under conditions of hydrological shortage and are able to induce transcription of heterologous genes in plants exposed to drought without negatively affecting the important agronomical characteristics of the plant so that plants that can grow under conditions of hydrological shortage with desired phonotypical characteristics are obtained.

We provide promoter nucleotide sequences which permit expression of a gene of interest in tissues of a plant when it is in conditions of hydrological shortage caused by drought or ground salinity. The promoter sequences comprise those that exhibit at least 80% identity with sequences or a portion of sequences of genes promoters Atlg05340 y Atlg80160 of Arabidopsis. Preferably, the sequences comprise sequences that exhibit at least 80% of identity with sequences or a portion of sequences SEQ ID N° 1 and SEQ ID N° 2 (See FIGS. 1 and 2, respectively).

“Identity %” means the percentage of identical nucleotides that can be easily calculated by those skilled in the art using software to compare sequences. Also, sequences and percentages of identity can be obtained using Internet resources such as BLAST (www.nebi.nlm.nih.gov) and FastDB program. Also, sequences that have 80% of identity can be defined as sequences that are hybridized with sequences SEQ ID N° 1 and SEQ ID N° 2 in conditions of strong astringency. Such conditions are presented in Sambrook et al. Molecular Cloning A Laboratory Manual (Cold Spring Harbor Press, 1989).

The sequences include the sequences or a portion of the sequences that are shown in FIG. 1 (SEQ ID N° 1) and FIG. 2 (SEQ ID N° 2).

We provide:
a) Obtaining a nucleotide sequence having the entire sequence or part of the sequence SEQ ID N° 1.
b) Obtaining a nucleotide molecule having the entire sequence or part of the sequence SEQ ID N° 1.
c) Obtaining an expression vector that contains any of the nucleotide sequences SEQ ID N° 1 and SEQ ID N° 2.
d) Obtaining microorganisms carriers of vector (c),
e) Transformation of plants with the vector in (c) or with the microorganism in (d), and
f) Obtaining vegetal cells, tissues, seeds or complete plants containing any of the sequences of invention SEQ ID N° 1 and SEQ ID N° 2, and where the plant is monocotyledon or dicotyledonous.

We use nucleotide sequences partially, totally, alone or in combination with other promoter sequences or promoter elements, use of expression vectors, transformed cells, and provide methods of obtaining transgenic plants of commercial interest.

### Identification and Isolation of Promoters

For the search of promoters and the subsequent functional evaluation, a first bibliographic analysis of transcriptomics studies published was carried out (Seki et al., 2001, Plant Cell 13(1): 61-72; Kreps et al., 2002, Plant Physiol. 130(4): 2129-2141), The search was orientated to tests of abiotic stress, specifically hydrological stress. Selected genes accomplished the following characteristics:
1. To be induced by treatment of hydrological shortage in vegetative tissue;
2. With positive response to other treatments of abiotic stress (osmotic and salinity)
3. To be induced by ABA in vegetative tissues and/or to be expressed in tissues which undergo natural desiccation (seeds).

To obtain promoter regions of the selected genes, genomic sequences for each locus described in the database TAIR (http://www.arabidopsis.org) were obtained. Specific Primers were designed using the software Primer 3 plus Untergasser et al., 2007, Nucleic Acids Research 35(suppl 2): W71-W74), to amplify through a chain reaction of polymerase (PCR) the totality of the promoter region of every gene using genomic DNA of Arabidopsis obtained with the method of Doyle and Doyle (1987) as template. Primers designed hybridize the extremes of intergenic regions, without including the reading frame open of each gene, but including the recognition site of ARA polymerase II, box TATA (5′-TATAAA-3)′.

Primers to obtain SEQ ID N° 1 were:

Fwd, 5′-AAGCTTTTTTGGAGGTAAGATATTAATTGCGC-3′
and
Rev, 5′-TCTAGATTAATGTAAAGAACTTTGATCTACTAAAGGC-3′.

Primers to obtain SEQ ID N° 2 were:

Fwd, 5′AATGAGTTATGTTGTAAGCTTCATCTAGCC-3′
and
Rev, 5′-TCTAGAGAGACGTACAGAAACAGAACGC-3′.

Both sequences were isolated by reaction of PCR where 100 ng DNA, 1.5 mM MgCl₂, 1 X Buffer PCR, 250 nM of each primer, 200 nM dNTPs and 0.25 U Taq polymerase (Invitrogen) were used. Parameters of the reaction of PCR used were: denaturation at 95% for 5 min, followed by 35 cycles at 95° C. for 30 s, 60° C. for 45 s and 72° C. for 60 s and finally an extension at 72° C. for 7 min. Products of PCR obtained, were separated through electrophoresis in agarose gel at 1% in buffer TBE 1X. The stripes obtained were cut and purified through E.Z.N.A® Gel extraction kit (Promega, Madison, Wis., EE.UU.) following the directions of the manufacturer. Isolated fragments were cloned in vector pGEM-T Easy System I (Promega) and the resulting plasmid was amplified in bacteria of E. coli DH5-alpha. Plasmids obtained containing the inserted fragment were sent to sequence the sequencing services of Macrogen Inc. (Chongro-ku, Seoul, Korea).

Through the use of databases useful to search regulating sequences in gene promoters of plants PlantCARE (Lescot et al., 2002, Nuc. Acid Res. 30(1): 325-327), PLACE (Higo et al., 1999, Nuc. Acid Res. 27(1): 297-300) and AGRIS (Davuluri et al., 2003, BMC Bioinfor. 4(1): 25) proximal sequences from both our promoters were analyzed. From this analysis, putative sites of union to transcription factors that respond to abiotic stress and ABA already described in the literature were found. For the promoter of SEQ ID N° 1 composed by 929 base pairs (pb), three boxes of response to ABA (ABRES, PyACGTGG/TC), two CRT/DRE (TACCGACAT) and one NACR (CATGTG) were identified.

The study of transgenic plants obtained by stable and transitory transformation of plants has permitted the basal promoter activity of promoters, as well as genes over-expression of interest that codify for proteins (beta-glucuronidase and VTE2/HPT1) in stress situations. The results observed in FIGS. 4 and 7 clearly show that our promoters have a low basal promoter activity and strongly increased when transgenic plants containing this promoter and codifying gene of interest are exposed to conditions of hydrological stress.

### BRIEF DESCRIPTION OF THE DRAWINGS

FIG. 1 shows SEQ ID N° 1.

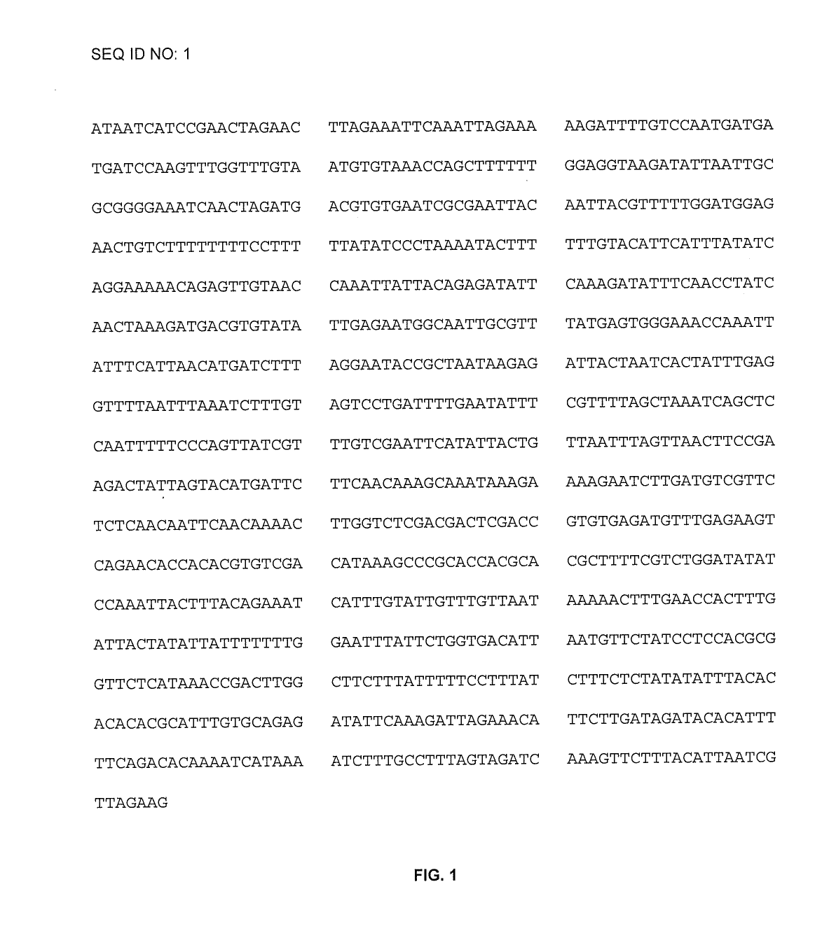

FIG. 2 shows SEQ ID N° 2.

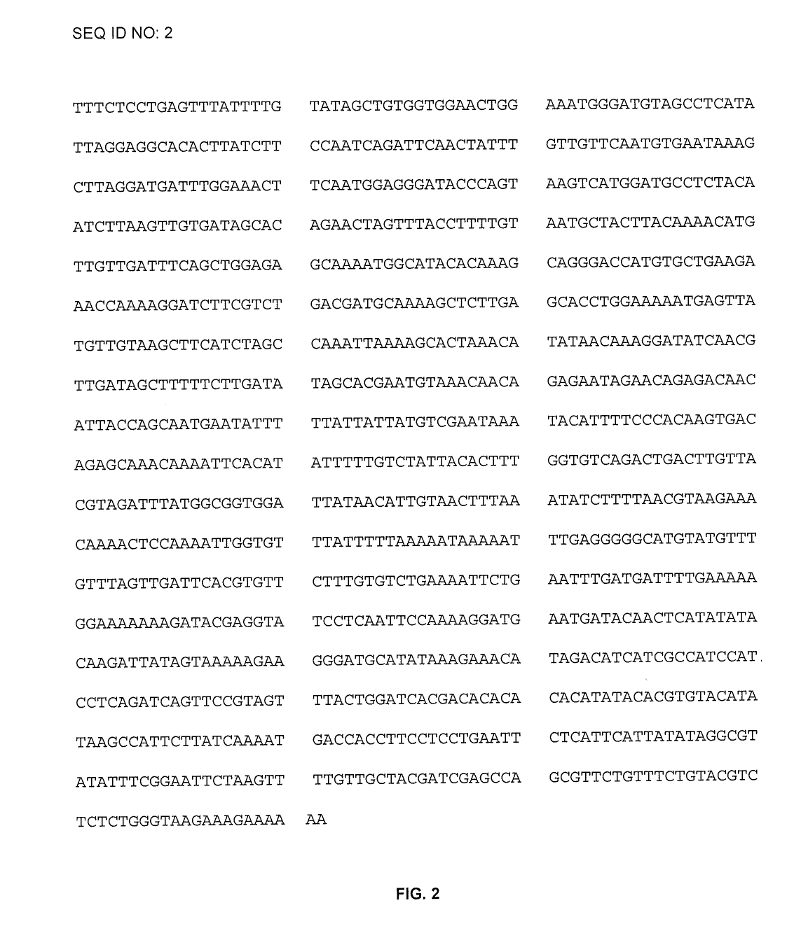

FIG. 3 shows constructions with promoters of SEQ ID N° 1 and SEQ ID N° 2 used in tests of transitory expression.

FIG. 4 shows a functional analysis of our promoter sequences.

FIG. 5 shows expression of ScVTE2.1 and alpha-tocopherol content induced by drought in transgenic plants.

FIG. 6 shows a hydrological state and assimilation rate of CO₂ in transgenic tobacco plants that express ScVTE2.1.

FIG. 7 shows symptoms of damage induced by hydrological stress in transgenic tobacco plants.

### DETAILED DESCRIPTION

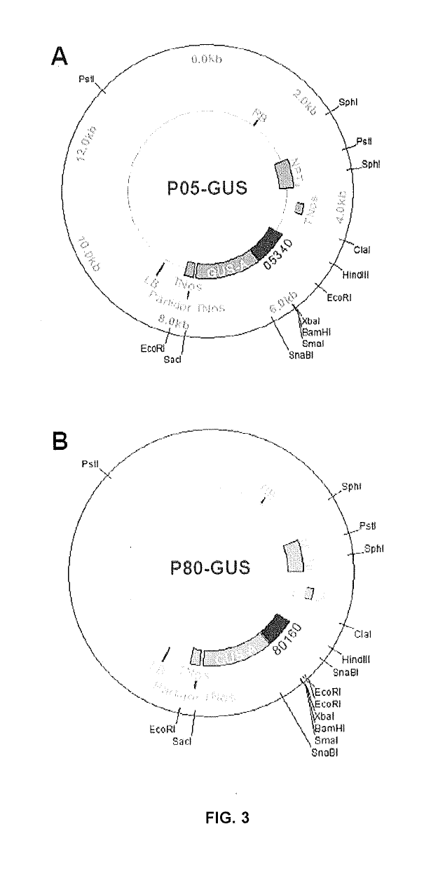

In FIG. 3, constructions with promoters of SEQ ID N° 1 and SEQ ID N° 2 used in tests of transitory expression are shown, where: (A) Construction map of P05-GUS, which contains the sequence of reporter gene GUS and promoter of SEQ ID N° 1 that was inserted in vector pBI121 replacing promoter 35S, and (B) Construction map of P80-GUS, which contains the sequence of reporter gene GUS and the promoter of SEQ ID N° 2 that was inserted in vector pBI121 replacing promoter 35S.

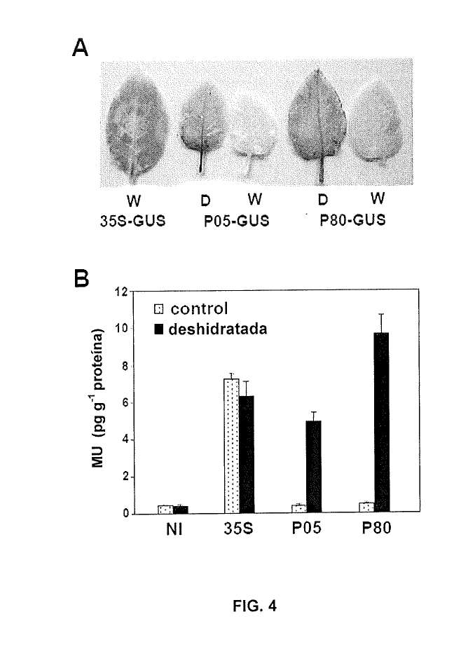

In FIG. 4, the functional analysis of promoter sequences of the invention is presented, where in FIG. 4A the histochemical analysis showing beta-glucoronidase activity is observed representing the activation of promoters in tobacco leaves transformed transitorily with constructions 35S-GUS, P05-GUS, P80-GUS. The leaves were separated and dehydrated (D) for 48 hours before staining or kept in plants with irrigation (W). FIG. 4B shows quantification of the same experiment described in FIG. 4A, through fluorometry. Bars represent averages of activity GUS±E.S. (n=4).

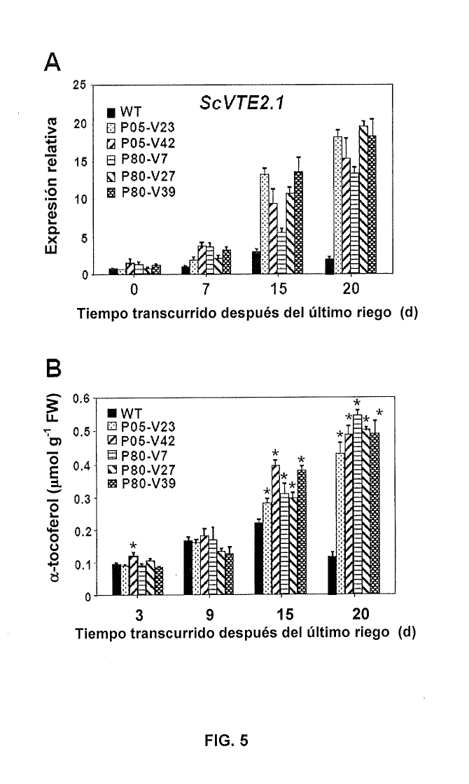

In FIG. 5, expression of ScVTE2.1 and content of alpha-tocopherol induced by drought in transgenic plants are shown, where in FIG. 5A the expression relative to transgene ScVTE2.1 is shown under control of promoters P05 and P80 in wild plants (WT) and transgenic lines of tobacco. The expression analysis was carried out through qRT-PCR using total ARN of plant subjected to suspension of irrigation for 0, 7, 15 and 20. Bars represent averages of activity GUS±E.S. (n=4). In FIG. 5B, concentration of alpha-tocopherol in tobacco leaves in the same test is shown. Bars represent activity averages GUS±E.S. (n=4). Asterisks denote significant differences regarding to WT (p<0.05).

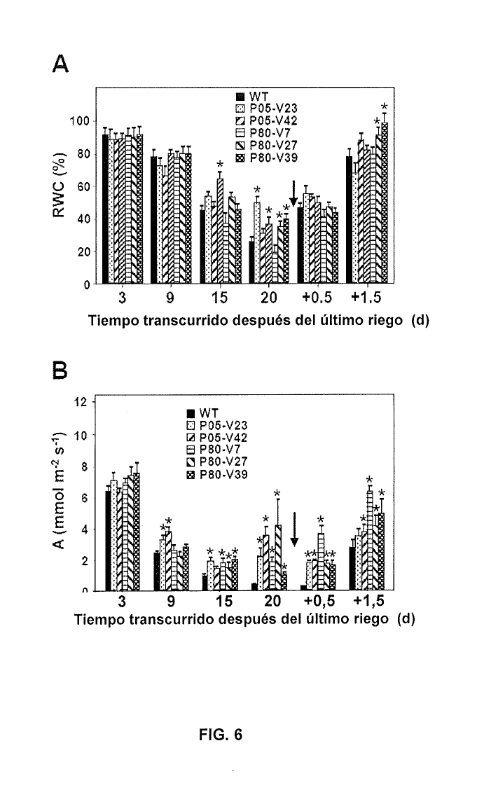

In FIG. 6, hydrological state and assimilation rate of CO₂ in transgenic tobacco plants that express ScVTE2.1 are shown, wherein the relative water content (RWT) is shown in FIG. 6A and in FIG. 6B the assimilation rate of CO₂ in wild and transgenic tobacco plants that express gene ScVTE2.1 is shown under control of promoters inducible by drought P05 and P80 and subjected to suspension of irrigation. Measurements were done in the third leaf of each plant 3, 9, 15 and 20 days after the last irrigation and 12 and 36 hours after the recovery through daily irrigation (indicated by the arrow). Bars represent averages of activity GUS±E.S. (n=4). Asterisks denote differences significant regarding to WT (p<0.05).

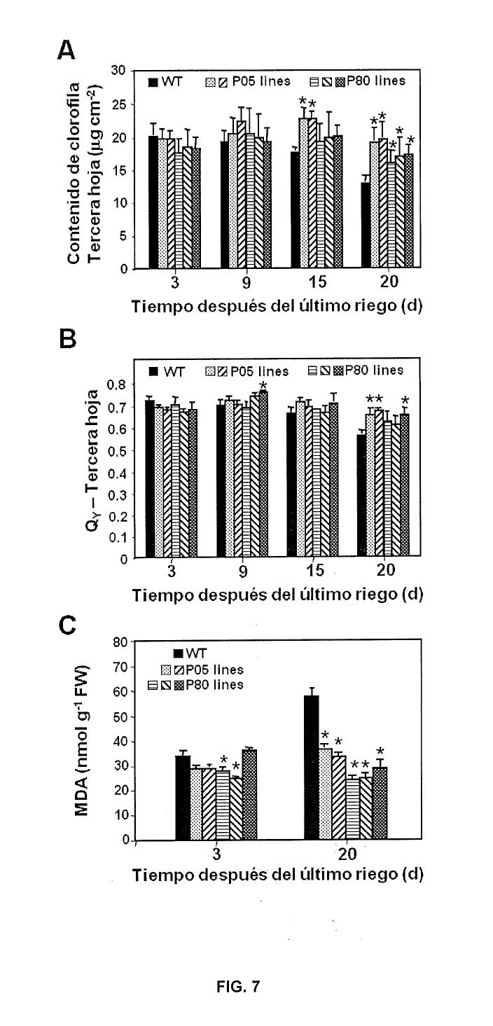

In FIG. 7, symptoms of induced damage by hydrological stress in transgenic tobacco plants are observed, where in FIG. 7A total content of chlorophyll is shown, in FIG. 7B quantum yield of PSII (Qy) is shown and FIG. 7C the content of malondialdehyde (MDA) in wild transgenic tobacco plants leaves (WT) that express gen Scvte2.1 is shown under control of inducible promoters by drought P05 and P80 and subject to suspension of irrigation. Measurements were carried out in the third leaf of each plant at 3, 9, 15 and 20 days after the last irrigation and 12 and 36 hours after the recovery through daily irrigation (indicated by an arrow). Bars represent averages of activity GUS±E.S. (n=6 for A y B; n=4 for C). Asterisks denote significant differences regarding to WT (p<0,05). Legends of bands are the same than in the previous figure.

### RESULTS

Inducibility of promoter sequences was demonstrated in two types of tests.

### 1. Inducibility of Promoter Sequences Through Transitory Transformation of Vegetal Tissue.

Every amplified promoter was cloned in the expression vector pBI121 upstream of beta-glucoronidase reporter gene sequence (GUS). The constructions were confirmed by restriction map and automatic sequencing of DNA to verify the right insertion of every promoter in the expression vector. Examples of genetic constructions that can be generated are shown in FIG. 3. Constructions obtained were introduced in Agrobacterium tumefasciens (strain LBA4404) by electroporation. Bacteria were cultured in Petri dishes with solid medium YM (0.04% yeast extract, 1% mannitol, 1,7 mM NaCl, 0.8 mM MgSO₄×7H₂O, 2.2 mM K₂HPO₄ and 15 gr/L agar) in the presence of antibiotics streptomycine (50 μL/dL), rifampicin (50 μL/dL) and kanamycin (50 μL/dL) and grown at 28° C. for 48 hours. For each construction, an isolated colony of Agrobacterium was selected, which was grown in liquid culture (3 ml) in YM medium with the same antibiotics in a stirrer (200 rpm) at 28° C. for 24 hours. Every mini-culture was used to inoculate a culture of 50 ml LB medium, and grown in stirring at 28° C. for 24 hours until it reached an optimal density OD60 0.6. Later, the culture was centrifuged at 4.500 g for 10 minutes to precipitate cells and re-suspended in an infiltration buffer (20 mM buffer MES pH 5.5, 0.5% glucose and 10 mM MgSO₄, 0.1 mM acetosyringone 0.02% Silwet L-77) (Yañez et al., 2009, Plant Cell Rep 28: 1497-1507). 6-weeks old tobacco plants were selected for transitory transformation. Third and fourth leaves were agro-infiltrated, where they are incorporated manually through the axial zone of leaves; cultures were re-suspended in the infiltration buffer, using a 5 ml syringe (without needle) without affecting foliar tissue. Every leaf was completely infiltrated and then was marked for its subsequent identification. To determine the activation degree of promoters under conditions of hydrological shortage, some plants were subjected to severe dehydration, without substrate in culture chambers up to 48 hours post-infiltration and other maintained with irrigation as control.

GUS transgene transitory expression was evaluated through histochemical staining through fluorometric method. For histochemical staining of the infiltrated tissue, the transformed leaves were incubated with a solution containing X-Gluc (100 mM pH 7 sodium phosphate, 10 mM EDTA pH 8, 0.1% Triton X-100, 0.5 mM beta-Mercaptoethanol, 1 mg/ml 5-bromine-4chloro-3-indolyl-beta-D-glucoronic acid or X-Gluc) for 48 hours at 37° C. Then, the infiltrated tissue was treated with fixing solution (425 ml absolute ethanol, 425 ml distillated water, 50 ml glacial acetic acid, 100 ml formalin 37%) for 48 hours at 37° C. Then, the infiltrated tissue was treated with a fixing solution (425 ml absolute ethanol, 425 ml distilled water, 50 ml glacial acetic acid, 100 ml formalin 37%) for 15 minutes, and subsequently chlorophyll and other pigments were extracted with a solution 3:1 methanol: acetone, to avoid that these compounds impede formation of blue cromogenic compound product of GUS activity on X-Gluc. This solution was replaced until complete disappearance of green color on the tissue and the results were registered through digital photography (Mohamed et al., 2004, Helia 27(40): 25-40). For enzymatic measurement by fluorometry (according to Jefferson et al., 1987 EMBO J 6(13): 3901-3907), discs of infiltrated leaves were extracted, grinded in the presence of liquid nitrogen and homogenized in GUS extraction buffer (Na₂HPO₄/NaH₂PO₄ 50 mM, pH 7.0, beta-mercaptoethanol 10 mM, PVPP, Na₂EDTA 10 mM, sarcosyl 0.1% and triton X-100 0.1%), then centrifuged for 30 min at 4° C. The supernatant was transferred in a new tube and maintained in ice. To 40 μL supernatant was added 32 μL extraction buffer and 8 μL MUG. To stop reaction, this was taken to a final volume of 2 ml with Na₂CO₃ 0.2 M. Measurements were carried out in the fluorometer Victor 3 (Perkin Elmer). Wavelengths used were 365 nm excitation and 465 nm emission.

Results of these assays are shown in FIG. 4. Infiltrated leaves of control plants irrigated did not present higher activity GUS, while leaves infiltrated with constructs 35S-GUS, P05-GUS (containing as promoter SEQ ID N° 1) and P80-GUS (containing as promoter SEQ ID N° 2) presented GUS staining of higher intensity (FIG. 4A). Quantification of enzymatic activity correlating with GUS reporter gene activation was measured by fluorometry. Again, both promoters achieved reporter gene activation at similar levels to the constitutive promoter 35S (FIG. 4B). Moreover, promoter P80-GUS (containing as promoter SEQ ID N° 2) presented higher activity than constitutive promoter. These results are significant not only for the induction level produced by each promoter but also for the low basal level of activity in conditions of sufficient availability of water in plants.

2. Inducibility of Promoter Sequences Through Study in Transgenic Tobacco Plants Expressing a Gene for a Protein that Confers Tolerance to Stress.

A cDNA of isolated VTE2.1 gene of Solanum chilense (ScVTE2.1) was used. This gene codifies the homogentisic fitol transferase (HPT) enzyme responsible for condensation of aromatic homogentisic acid (HGA) precursor with diphosphate fitol (PDP), key step for synthesis of tocopherols in plants (Collakova et al., 2003, Plant Physiol 131(2): 632-642). Each genetic constructions were generated comprising the promoter sequences of promoters (SEQ ID N° 1 and SEQ ID N° 2) fused upstream of cDNA de ScVTE2.1 inside the transformation vector pBI121. Constructions obtained were confirmed by sequestration before its insertion in strain Agrobacterium tumefasciens (LBA4404) through electroporation. With this genetic construction, tobacco plants transformations though infection of leaves discs with A. Tumefasciens was executed using protocol described by Horsch et al (1986, Proc. Natl. Acad. Sci. USA 83(8):2571-5).

At least 50 sprouts regenerated for each event of transformation were selected and genomic DNA was selected to analyze the presence of transgene through PCR using specific dividers for gene ScVTE2.1 and selection gene NPTII present in T-DNA of vector pBI121. Those transgenic lines that amplified both sequences were multiplied. They grew in a greenhouse to obtain seeds until generation T2. To prove the efficiency of these promoters in activating the transgene (VTE2.1 in this case), some transgenic lines were selected in every construction to carry out stress tests cause by hydrological shortage with 8 to 10-weeks old plants that were irrigated up to 20 days. During these essays, the following measurements were carried out:
(a) Evaluation of expression level of transgene ScVTE2.1 through quantitative PCR in real time.

For this purpose, RNA was extracted though SV system total RNA (Promega), following recommendations of the manufacturer. 2 mg RNA was treated by DNase I (Ambion) before being used to synthesize cDNA in a reaction of 20 μL using AffinityScript QPCR cDNA Synthesis Kit (Stratagene, La Jolla, Calif., USA). PCR reaction was done with a system ABI Prism 7000 (Applied Biosystems) with the following parameters: 95° C. for 10 min, then 40 cycles of 95° C. during 15 s, 60° C. for 15 s and 72° C. for 20 s. Each reaction contained 10 μL SYBR Green Master Mix (Stratagene) and 0.25 μM of every specific primer. Tobacco ribosomal L25 gene (L18908) was used as a control gene of constitutive expression. Primers used for amplification were:

ScVTE2-Fwd, 5′-GAGTTTTTGGCTTGGATGGA-3′
and
ScVTE2-Rev, 5′TATCACCGCTCGTACAGCAA-3′;
NtL25-Fwd, 5′-AGGCTGTCAAGTCAGGATCAAC-3′
and
NtL25-Rev 5′-ATTGCAGACTCTGTGGTGAGG-3′.

The reaction specificity was verified by dissociation curve analysis and relative quantification through comparative method ΔCt.

FIG. 5 shows that transgene expression increased in transgenic lines transformed with constructions P05-GUS (containing as promoter SEQ ID N° 1) and P80-GUS (containing as promoter SEQ ID N° 1)
(b) Tocopherols measurement: Tocopherols were extracted grinding 500 mg of fresh leaves in presence of liquid nitrogen, 50 μL tocopherol acetate were added as internal standard, as well as 0.1 g ascorbic acid and citric acid as antioxidants. The macerated composition was re-suspended in methanol and the extracts were homogenized with Ultra Turrax (IKA Werke, Staufen, Germany) and centrifuged. The supernatant was recovered in a new tube to extract completely the solvent under vacuum in a rotavapor (Speed-vac, Jouan, Saint Herblain, France). Then, the samples were re-suspended in methanol and sieved using a filter of 5 μL. For quantification, high-pressure liquid chromatography equipment Agilent serie 1100 was used; the chromatography separation was carried out in reverse phase on a C18 column (Kromasil 100 C18 5 μm 100×2.0 mm, Scharlau). As mobile phase, mixture of methanol was used: acetonitrile (80:20 v/v). Detection was obtained with excitation of 292 nm and an emission of 330 nm. 20 μL of sample were injected with three repetitions for each extract. Quantification was done using a calibration curve using alpha- and gamma-tocopherol as standard. FIG. 5B shows that concentration of alpha-tocopherol increased in transgenic lines transformed with constructions P05-GUS (containing SEQ ID N° 1 as promoter) and p80-GUS (containing SEQ ID N° 2 as promoter) from 3 to 5 times the concentration of tocopherol after 20 days of initiated the stress treatment in comparison to wild tobacco.

Also, parameters of tolerance to hydrological shortage in the same plants of previous assays were measured to correlate them with the higher expression of transgene product of the action of promoter regions. For this purpose the following were measured:
(a) Relative water content: it was determined according to equation: RWC=(PF−PS/PH−PS)×100, where: PF corresponds to the fresh weight of the leaf when collecting the tissue; PH, corresponds to the weight of the leaf after 24 hours of hydration in distilled water and PS corresponds to the dry weight alter 24 hours of drying at 60° C.
(b) Quantum performance of PS II: it was measured by emission of chlorophyll fluorescence and this parameter was used as an indicator of damage to photosynthetic apparatus. Fluorescence of chlorophyll was measured with a portable fluorometer Fluorpen (PAM-2000, Walz, Effeltrich, Germany). Efficiency of quantum performance of PSII was calculated by QYPSII=(Fm 0−Ft)/Fm 0 (Genty et al., 1989, Biochim Biophys Acta 990(1): 87-92). Six measurements were done during the morning, in the third and fourth leaf of nine leaves of every plant.
(c) Chlorophyll determination: to determine chlorophyll content of leaves, a chlorophyll meter CCM-200 (Opti-Sciences, Inc., Tyngsboro, Mass.) was used. Readings were carried out in the third and fourth leaves of plants under hydrological stress for 15 days in the absence of irrigation. Three leaves of every genotype were used.
(d) Determination of lipid peroxidation: Levels of lipid peroxidation were also measured as indicators of oxidative damage in membranes caused by hydrological shortage in the vegetal tissue. This is done directly through the evolution of the malonaldehyde formation, according to the technique described by Esterbauer and Cheeseman (Esterbauer y Cheeseman 1990, Methods in Enzymology 186: 407-421). 200 mg of fresh tobacco leaves were macerated in 5 m l ethanol at 80%, the samples were homogenized through the use of Ultra Turrax for 1 minute and centrifuged at 400 RPM 15 min.

The supernatant was divided in 15 ml two tubes, one with trichloroacetic acid 5% w/v (TCA) at 4° C. and the other with a volume of thiobarbituric acid 0.67% w/v %, both were incubated at 100° C. during 60 minutes and immediately cooled in ice for 15 min. Subsequently, absorbance of every sample in a spectrophotometer was measured (Genesys 10 UV: Thermo Electron Corporation) at 532 nm and 600 nm. Three biological replicas were analyzed in every sampled point.

Tocopherols or Vitamin E are considered effective antioxidants for protection of lipid membranes. Tocopherol synthesis localized in photosynthetic membranes and accumulation of these in plants in response to stress suggests that tocopherols play an important role in photosynthetic organisms (Falk y Munné-Bosch, 2010, J Exp Bot 61(6): 1549-1566; Munne-Bosch and Alegre, 2002, Crit. Rev. Plant Sci. 21(1): 31-57; Collakova and DellaPenna, 2003, Plant Physiol. 131(2): 632-642).

Several studies with plants that overexpress genes of tocopherols synthesis route have shown to have a better capacity to tolerate stress caused by cold and salinity, less damage at a cell level and better physiological state in general. For example, it has been observed that when overexpressing in tobacco the second transfearase of metabolic route VTE1, these plants presented a higher accumulation of tocopherol, higher water relative content, higher chlorophyll content and parameter that evidence oxidative damage as accumulation of electrolytes, hydrogen peroxide and malonaldehyde, decreased in comparison to plants without transformation and to transgenic lines with accumulation of tocopherols (Liu et al., 2008, Biotechnol. Lett. 30 (7): 1275-1280). Overexpression of VTE2.1 in Arabidopsis increased 4 times the content of tocopherol in leaves (Collakova and DellaPenna 2003, Plant Physiol. 131(2): 632-642).

On the other hand, mutants for genes of this route are more susceptible to damage caused by stress. In seeds and seedlings of mutant Arabidopsis lacking of tocopherol, an increase of lipid peroxidation has been observed, which implicates a diminution in the accumulation of linoleic acid and an increase in toxics derived from malonaldehyde, with this, the longevity of seeds is reduced and the development of seedlings is affected. (Sattler et al., 2004, Plant Cell 16(6): 1419-1432). VTE1 gene silencing also important for tocopherols synthesis produce a reduction of 95% of alpha-tocopherol and plants may exhibit a reduced damage by the application of sorbitol in the substrate or by the application of methyl viologen (Abbasi et al., 2007, Plant Physiol 143(4): 1720-1738)

Plants generated with promoters of the present invention and gene ScVTE2.1 presented:
(1) Less reduction of relative water content (RWC) than in wild plants. FIG. 6A shows that transgenic lines keep more elevated values of RWC than wild ones (WT) and they improve faster after reposition of irrigation.
(2) Higher carbon fixation rate (FIG. 6B) than in wild plants, which is an indication that exists better use of water (biomass production) in conditions of stress probably because of the less damage to photosynthetic apparatus.
(3) Less reduction of quantum performance of photo-system II than in wild plants, which reflect less damage to photosynthetic apparatus of leaves (FIG. 7A).
(4) Less chlorophyll loss (FIG. 7B) than in wild plants, which suggests also a delay in senescence of leaves caused by drought.
(5) Less production of MDA (FIG. 7C) than in wild plants, which suggests that exist less damage of lipids of membrane product of oxidative stress generated by drought.

1. A promoter nucleotide sequence permitting regulation of gene expression in plants, wherein said sequence comprises at least 80% of identity with sequence or a portion of the promoter sequence of genes Atlg05340 or Atlg80160 of Arabidopsis.

2. The promoter nucleotide sequence according to claim 1, wherein said sequence regulates gene transcription of interest so that it presents a low basal expression in optimal conditions of water resources for plants and presents an activation of its promoter activity in conditions of hydrological shortage.

3. The promoter nucleotide sequence according to claim 1, wherein said sequence regulates gene transcription of interest so that it presents an activation of its promoter activity in conditions of hydrological shortage caused by drought or ground salinity.

4. The promoter nucleotide sequence according to claim 1, wherein said sequence presents at least 80% of identity with sequence or a portion of sequence SEQ ID N° 1 or the sequence SEQ ID N° 2.

5. The promoter nucleotide sequence according to claim 1, wherein said sequence comprises sequence SEQ ID N° 1.

6. The promoter nucleotide sequence according to claim 1, wherein said sequence comprises sequence SEQ ID N° 2.

7. A vector comprising a promoter nucleotide sequence comprising at least 80% of identity with sequence or a portion of promoter sequence of genes Atlg05340 or Atlg80160 of Arabidopsis.

8. The vector according to claim 7, comprising a promoter nucleotide sequence that presents at least 80% of identity with sequence or a portion of sequence SEQ ID N° 1 or the sequence SEQ ID N° 2.

9. A microorganism comprising the vector of claim 7.

10. A method of obtaining a plant genetically modified comprising:
a) providing a nucleotide molecule comprising at least 80% of identity with sequences or a portion of promoter sequence of genes Atlg05340 or Atlg80160 of Arabidopsis;
b) providing an expression vector containing any of the promoter sequences of genes Atlg05340 or Atlg80160 of Arabidopsis;
c) optionally providing a microorganism carrier of the vector;
d) transforming plants with the vector; and
e) providing vegetal cells, tissues, seeds or complete plants containing any of the sequences or a portion of promoter sequence of genes Atlg05340 or Atlg80160 of Arabidopsis, and where the plant is monocotyledon or dicotyledonous.

11. The method according to claim 10, wherein the promoter sequence of Atlg05340 or Atlg80160 of Arabidopsis comprises the entire sequence or part of sequence SEQ ID N° 1 or sequence SEQ ID N° 2.

12. The method according to claim 10, comprising also the obtaining of a microorganism carrier of vector (b);
a) transforming the plant with microorganism of (c); and
b) obtaining vegetal cells, tissues, seeds or complete plants containing any of the promoter sequences, where the plant is monocotyledon or dicotyledonous.

13. A method of obtaining promoter sequences of genes Atlg05340 or Atlg80160 of Arabidopsis comprising:
obtaining genomic sequences for each locus of genes Atlg05340 or Atlg80160 of Arabidopsis; designing specific primers to amplify through chain reactions polymerase (PCR) of the entire promoter region of each gene using genomic DNA of Arabidopsis, where primers hybridized the extremes of intergenic regions, without including the reading frame open to each gene but including the recognition site of ARN polymerase II, box TATA (5′-TATAAA-3).

14. The method according to claim 13, wherein the genomic sequence for gene of locus Atlg05340 of Arabidopsis comprises promoter sequence of all or part of SEQ ID N° 1.

15. The method according to claim 13, wherein the genomic sequence for gene of locus Atlg05340 of Arabidopsis comprises promoter sequence of the entire sequence or part of the SEQ ID N° 2.

16. The method according to claim 13, wherein primers to obtain of SEQ ID N° 1 comprise:
(SEQ ID NO: 3)
Fwd, 5′-AAGCTTTTTTGGAGGTAAGATATTAATTGCGC-3′
and
(SEQ ID NO: 4)
Rev, 5′-TCTAGATTAATGTAAAGAACTTTGATCTACTAAAGGC-3′.

17. The method according to claim 13, wherein primers to obtain of SEQ ID N° 2 comprise:
(SEQ ID NO: 5)
Fwd, 5′AATGAGTTATGTTGTAAGCTTCATCTAGCC-3′
and
(SEQ ID NO: 6)
Rev, 5′-TCTAGAGAGACGTACAGAAACAGAACGC-3′.


SEQUENCE LISTING (10 sequences)

SEQ ID NO: 1 - DNA, 1027 bp, Arabidopsis thaliana
ataatcatccgaactagaacttagaaattcaaattagaaaaagattttgtccaatgatgatgatccaagtttggtttgtaatgtgtaaaccagcttttttggaggtaagatattaattgcgcggggaaatcaactagatgacgtgtgaatcgcgaattacaattacgtttttggatggagaactgtcttttttttcctttttatatccctaaaatacttttttgtacattcatttatatcaggaaaaacagagttgtaaccaaattattacagagatattcaaagatatttcaacctatcaactaaagatgacgtgtatattgagaatggcaattgcgtttatgagtgggaaaccaaattatttcattaacatgatctttaggaataccgctaataagagattactaatcactatttgaggttttaatttaaatctttgtagtcctgattttgaatatttcgttttagctaaatcagctccaatttttcccagttatcgtttgtcgaattcatattactgttaatttagttaacttccgaagactattagtacatgattcttcaacaaagcaaataaagaaaagaatcttgatgtcgttctctcaacaattcaacaaaacttggtctcgacgactcgaccgtgtgagatgtttgagaagtcagaacaccacacgtgtcgacataaagcccgcaccacgcacgcttttcgtctggatatatccaaattactttacagaaatcatttgtattgtttgttaataaaaactttgaaccactttgattactatattatttttttggaatttattctggtgacattaatgttctatcctccacgcggttctcataaaccgacttggcttctttatttttcctttatctttctctatatatttacacacacacgcatttgtgcagagatattcaaagattagaaacattcttgatagatacacatttttcagacacaaaatcataaaatctttgcctttagtagatcaaagttctttacattaatcgttagaag

SEQ ID NO: 2 - DNA, 1102 bp, Arabidopsis thaliana
tttctcctgagtttattttgtatagctgtggtggaactggaaatgggatgtagcctcatattaggaggcacacttatcttccaatcagattcaactatttgttgttcaatgtgaataaagcttaggatgatttggaaacttcaatggagggatacccagtaagtcatggatgcctctacaatcttaagttgtgatagcacagaactagtttaccttttgtaatgctacttacaaaacatgttgttgatttcagctggagagcaaaatggcatacacaaagcagggaccatgtgctgaagaaaccaaaaggatcttcgtctgacgatgcaaaagctcttgagcacctggaaaaatgagttatgttgtaagcttcatctagccaaattaaaagcactaaacatataacaaaggatatcaacgttgatagctttttcttgatatagcacgaatgtaaacaacagagaatagaacagagacaacattaccagcaatgaatatttttattattatgtcgaataaatacattttcccacaagtgacagagcaaacaaaattcacatatttttgtctattacactttggtgtcagactgacttgttacgtagatttatggcggtggattataacattgtaactttaaatatcttttaacgtaagaaacaaaactccaaaattggtgtttatttttaaaaataaaaatttgagggggcatgtatgtttgtttagttgattcacgtgttctttgtgtctgaaaattctgaatttgatgattttgaaaaaggaaaaaaagatacgaggtatcctcaattccaaaaggatgaatgatacaactcatatatacaagattatagtaaaaagaagggatgcatataaagaaacatagacatcatcgccatccatcctcagatcagttccgtagtttactggatcacgacacacacacatatacacgtgtacatataagccattcttatcaaaatgaccaccttcctcctgaattctcattcattatataggcgtatatttcggaattctaagttttgttgctacgatcgagccagcgttctgtttctgtacgtctctctgggtaagaaagaaaaaa

SEQ ID NO: 3 - DNA, 32 bp, Artificial Sequence
  Fwd Primer
aagcttttttggaggtaagatattaattgcgc

SEQ ID NO: 4 - DNA, 37 bp, Artificial Sequence
  Rev Primer
tctagattaatgtaaagaactttgatctactaaaggc

SEQ ID NO: 5 - DNA, 30 bp, Artificial Sequence
  Fwd Primer
aatgagttatgttgtaagcttcatctagcc

SEQ ID NO: 6 - DNA, 28 bp, Artificial Sequence
  Rev Primer
tctagagagacgtacagaaacagaacgc

SEQ ID NO: 7 - DNA, 20 bp, Artificial Sequence
  ScVTE2-Fwd Primer
gagtttttggcttggatgga

SEQ ID NO: 8 - DNA, 20 bp, Artificial Sequence
  ScVTE2-Rev Primer
tatcaccgctcgtacagcaa

SEQ ID NO: 9 - DNA, 22 bp, Artificial Sequence
  NtL25-Fwd Primer
aggctgtcaagtcaggatcaac

SEQ ID NO: 10 - DNA, 21 bp, Artificial Sequence
  NtL25-Rev Primer
attgcagactctgtggtgagg